(19) United States
(12) Patent Application Publication
(10) Pub. No.: US 2020/0401214 A1
(43) Pub. Date: Dec. 24, 2020

(54) SYSTEMS FOR MONITORING AND ASSESSING PERFORMANCE IN VIRTUAL OR AUGMENTED REALITY

(71) Applicant: XR Health IL LTD, Tel Aviv (IL)

(72) Inventors: Eran Orr, Brookline, MA (US); Miki Levy, Misgav Dov (IL); Omer Weissberger, Even-Yehuda (IL)

(21) Appl. No.: 17/013,016

(22) Filed: Sep. 4, 2020

Related U.S. Application Data
(63) Continuation of application No. PCT/US2019/021439, filed on Mar. 8, 2019.
(60) Provisional application No. 62/640,420, filed on Mar. 8, 2018.
(60) Provisional application No. 62/646,569, filed on Mar. 22, 2018.
(60) Provisional application No. 62/652,714, filed on Apr. 4, 2018.

(51) Int. Cl. G06F 3/01; H04L 29/06; G06T 7/73; G06T 11/00; G16H 40/67; G16H 20/30

(52) U.S. Cl. CPC G06F 3/011; H04L 67/38; G06T 7/75; G06T 2200/24; G16H 40/67; G16H 20/30; G06T 2210/41; G06T 11/00

(57) ABSTRACT

Provided herein are methods of and computer program products for physical therapy using VR/AR, specifically, for guiding user motion for physiotherapy in VR/AR environments. In various embodiments, a virtual environment is provided to a user via a VR/AR system. An event marker is provided at a first location within the virtual environment. A position of the event marker is adjusted to a second location. Positional data is collected based on the user's interaction with the one or more event markers. The positional data is provided to a remote server via a network and a compliance metric is determined based on the positional data. When the compliance metric differs from a predetermined range, an adjustment is applied to the event marker. In various embodiments, a visual field of a user may be altered and the user guided to repeat a task to assess and/or monitor proprioception.

### CROSS-REFERENCE TO RELATED APPLICATIONS

This application claims the benefit of U.S. Provisional Patent Application No. 62/640,420, filed on Mar. 8, 2018, U.S. Provisional Patent Application No. 62/646,569, filed on Mar. 22, 2018, and U.S. Provisional Patent Application No. 62/652,714, filed on Apr. 4, 2018, each of which is incorporated by reference in its entirety.

### BACKGROUND

Embodiments of the present disclosure relate to monitoring and assessing user performance of rehabilitation activities in virtual reality (VR) or augmented reality (AR) environments.

### BRIEF SUMMARY

According to embodiments of the present disclosure, methods of and computer program products for monitoring and assessing performance while immersed in a virtual or augmented reality are provided. In various embodiments, a virtual environment is provided to a user via a VR/AR system. An event marker is provided at a first location within the virtual environment. A position of the event marker is adjusted to a second location. Positional data is collected based on the user's interaction with the one or more event markers. The positional data is provided to a remote server via a network and a compliance metric is determined based on the positional data. When the compliance metric differs from a predetermined range, an adjustment is applied to the event marker.

In various embodiments, the event marker includes a visual object displayed within the virtual or augmented reality environment. In various embodiments, the method further includes adjusting the position of the event marker to a third location based on the applied first adjustment. In various embodiments, the first adjustment includes a speed of motion of the event marker as the position of the event marker is adjusted. In various embodiments, the first adjustment includes a slower speed. In various embodiments, the first adjustment includes a faster speed. In various embodiments, the first adjustment includes a change in distance of the event marker as the position of the event marker is adjusted. In various embodiments, the first adjustment includes a second distance that is greater than the first distance. In various embodiments, the first adjustment includes a second distance that is less than the first distance. In various embodiments, the first adjustment includes an increase in a number of repetitions of the user interaction with the event marker. In various embodiments, the first adjustment includes a decrease in a number of repetitions of the user interaction with the event marker.

According to embodiments of the present disclosure, systems for, methods of, and computer program products for assessing and practicing proprioception in virtual reality or augmented reality environments are disclosed. In various embodiments, a virtual environment is provided to a user via a virtual or augmented reality system. The user is guided to perform a task involving movement of a body part of the user via the virtual or augmented reality environment, wherein guiding the user to perform the task comprises displaying a visual object to the user. A first set of data including positional data of the body part is collected based on the user's performance of the task. A visual field of the user is altered within the virtual or augmented reality environment. The user is guided to repeat the task with the altered visual field in the virtual or augmented reality environment. A second set of data including positional data of the body part is collected based on the user's performance of the task with the altered visual field. The first set of data and the second set of data are provided to a remote server via a network. A compliance metric is determined based on the first set of data and the second set of data. When the compliance metric differs from a predetermined range, an adjustment is applied to the task.

In various embodiments, altering the visual field includes removing the visual object displayed in connection with the task. In various embodiments, altering the visual field includes blacking out the visual field of the user. In various embodiments, the visual field is blacked out in its entirety. In various embodiments, altering the visual field comprises partially obstructing the visual field of the user. In various embodiments, applying a first adjustment to the task includes increasing an amount of time the visual object is displayed to the user when guiding the user to perform the task involving movement of a body part. In various embodiments, applying a first adjustment to the task includes decreasing an amount of time the visual object is displayed to the user when guiding the user to perform the task involving movement of a body part.

According to embodiments of the present disclosure, systems for, methods of, and computer program products for closed circuit assessment, decision-making, and protocol rendering in virtual reality or augmented reality environments are disclosed. In various embodiments, a virtual environment is provided to a user via a virtual or augmented reality system. The virtual environment includes an avatar using machine learning or artificial intelligence to communicate with the user. Screening data is collected from the user's interaction with the avatar in the virtual environment. A customized evaluation, training, or treatment protocol is determined for the user based at least in part on the screening data. The user is guided to perform a task in the evaluation, training, or treatment protocol via the virtual or augmented reality system. Data is collected from a plurality of sensors relating to the user's performance of the task. The collected data is analyzed and a report is generated based on the user's performance of the task.

### BRIEF DESCRIPTION OF THE SEVERAL VIEWS OF THE DRAWINGS

FIG. 1 illustrates an exemplary virtual reality headset according to embodiments of the present disclosure.

FIG. 2 illustrates an exemplary system according to embodiments of the present disclosure.

FIG. 3 illustrates an exemplary cloud service according to embodiments of the present disclosure.

FIG. 4 illustrates the Torso Sway Index (TSI) and Head Sway Index (HSI) of a person according to embodiments of the present disclosure.

FIG. 5 illustrates a method of sway assessment according to embodiments of the present disclosure.

FIGS. 6A-D illustrate exemplary user motion according to embodiments of the present disclosure.

FIG. 7 illustrates a method of guiding user motion according to embodiments of the present disclosure.

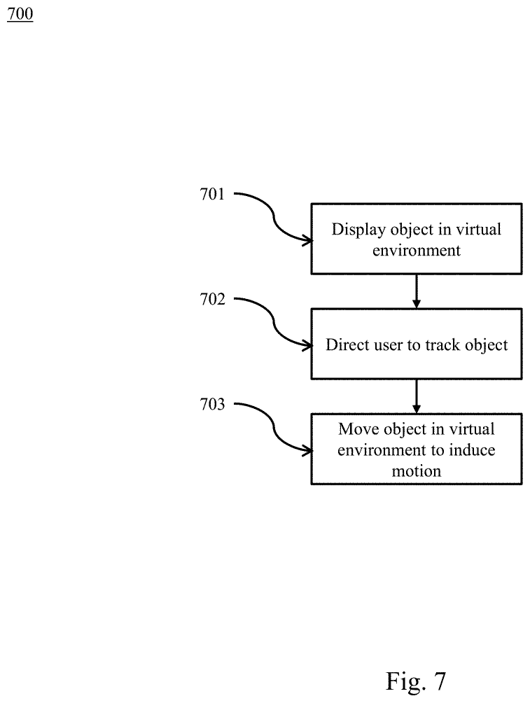

FIG. 8 illustrates tracking data according to embodiments of the present disclosure.

FIG. 9 illustrates a method of tracking data according to embodiments of the present disclosure.

FIG. 10 illustrates degrees of freedom on various joints in an exemplary human kinematic model according to embodiments of the present disclosure.

FIG. 11 illustrates an exemplary process for assessing and practicing proprioception in virtual reality or augmented reality environments according to embodiments of the present disclosure.

FIG. 12 illustrates an exemplary technique of determining deviation between actual and required positions according to embodiments of the present disclosure.

FIG. 13 illustrates an example of a procedure in which the patient performs a controlled neck movement, creating a trail in the virtual environment with a FIG. 8 shape according to embodiments of the present disclosure.

FIG. 14 is a flow chart illustrating an exemplary method of assessing and practicing proprioception in virtual reality or augmented reality environments according to embodiments of the present disclosure.

FIG. 15 is a flow chart illustrating an exemplary method for closed circuit assessment, decision-making, and protocol rendering in virtual reality or augmented reality environments according to embodiments of the present disclosure.

FIG. 16 depicts a computing node according to embodiments of the present disclosure.

### DETAILED DESCRIPTION

Physical therapy attempts to address the illnesses or injuries that limit a person's abilities to move and perform functional activities in their daily lives. Physical therapy may be prescribed to address a variety of pain and mobility issues across various regions of the body. In general, a program of physical therapy is based on an individual's history and the results of a physical examination to arrive at a diagnosis. A given physical therapy program may integrate assistance with specific exercises, manual therapy and manipulation, mechanical devices such as traction, education, physical agents such as heat, cold, electricity, sound waves, radiation, assistive devices, prostheses, orthoses and other interventions. Physical therapy may also be prescribed as a preventative measure to prevent the loss of mobility before it occurs by developing fitness and wellness-oriented programs for healthier and more active lifestyles. This may include providing therapeutic treatment where movement and function are threatened by aging, injury, disease or environmental factors.

As an example, individuals suffer from neck pain or need to perform neck exercises for various reasons. For example, people who have been involved in a motor vehicle accident or have suffered an injury while playing contact sports are prone to develop a whiplash associated disorder (WAD), a condition resulting from cervical acceleration-deceleration (CAD). It will be appreciated that this is just one of many potential injuries that may result in neck injury or pain necessitating rehabilitation.

The majority of people who suffer from non-specific neck pain (NSNP) may have experienced symptoms associated with WAD or have an undiagnosed cervical herniated disc. For this population, the recommended treatment regimen often includes a variety of exercises promoting neck movement and other functional activity training, leading to improved rehabilitation.

Poor adherence to treatment can have negative effects on outcomes and healthcare cost, irrespective of the region of the body affected. Poor treatment adherence is associated with low levels of physical activity at baseline or in previous weeks, low in-treatment adherence with exercise, low self-efficacy, depression, anxiety, helplessness, poor social support/activity, greater perceived number of barriers to exercise and increased pain levels during exercise. Studies have shown that about 14% of physiotherapy patients do not return for follow-up outpatient appointments. Other studies have suggested that overall non-adherence with treatment and exercise performance may be as high as 70%. Patients that suffer from chronic or other long-term conditions (such as those associated with WAD or NSNP) are even less inclined to perform recommended home training.

Adherent patients generally have better treatment outcomes than non-adherent patients. However, although many physical therapy exercises may be carried out in the comfort of one's home, patients cite the monotony of exercises and associated pain as contributing to non-adherence.

Irrespective of adherence, home training has several limitations. With no direct guidance from the clinician, the patient has no immediate feedback to confirm correct performance of required exercises. Lack of such guidance and supervision often leads to even lower adherence. As a result, the pain of an initial sensed condition may persist or even worsen—leading to other required medical interventions that could have been prevented, thus also increasing associated costs of the initial condition.

Accordingly, there is a need for devices, systems, and methods that facilitate comprehensive performance and compliance with physical therapy and therapeutic exercise regimens.

According to various embodiments of the present disclosure, various devices, systems, and methods are provided to facilitate therapy and physical training assisted by virtual or augmented reality environments.

Augment reality (AR) and virtual reality (VR) typically reproduce real world environments where users perform tasks in a way similar to real world experiences. AR/VR experiences allow users to climb virtual mountains, play virtual sports games, jump out of an airplane, shoot targets, and engage in other physically demanding real-world behavior. Since these experiences require real life—analog—skill (rather than computer game skill), there is great potential in harnessing user performance in AR or VR to assess and improve real life performance.

Some VR games may try to track user performance, but they lack a cross-platform multi-experience solution that tracks a user across all his or her AR or VR activities. Likewise, they do not provide measurement of medically useful parameters nor do they track wellness factors that can impact quality of life and drive a greater meaning into VR.

It will be appreciated that a variety of virtual and augmented reality devices are known in the art. For example, various head-mounted displays providing either immersive video or video overlay are provided by various vendors. Some such devices integrate a smart phone within a headset, the smart phone providing computing and wireless communication resources for each virtual or augmented reality application. Some such devices connect via wired or wireless connection to an external computing node such as a personal computer. Yet other devices may include an integrated computing node, providing some or all of the computing and connectivity required for a given application.

Virtual or augmented reality displays may be coupled with a variety of motion sensors in order to track a user's motion within a virtual environment. Such motion tracking may be used to navigate within a virtual environment, to manipulate a user's avatar in the virtual environment, or to interact with other objects in the virtual environment. In some devices that integrate a smartphone, head tracking may be provided by sensors integrated in the smartphone, such as an orientation sensor, gyroscope, accelerometer, or geomagnetic field sensor. Sensors may be integrated in a headset, or may be held by a user, or attached to various body parts to provide detailed information on user positioning.

In various embodiments, a mobile phone may be attached to the body of a user to thereby record motion data using components such as, for example, an internal gyroscope, internal accelerometer, etc.

In the course of a program of rehabilitation, patients follow physical training protocols that guide the physical aspect of their recovery and define what physical motions and activities are required for treatment. Such protocols often include repetitive motions and activities designed to activate and facilitate movement of specific body parts. The patient may be guided to follow and repeat these motions and activities through the assistance of external equipment (e.g., weights or bands) that can control resistance and difficulty.

As discussed above, traditional protocol training often exhibits low adherence. In many cases, low adherence may be attributed to the repetitive, unengaging nature of such protocols. To address this boredom, a user may watch a television screen while doing the motions and activities or listen to music. However, even with this additional stimulus, the motions and activities themselves continue to be tedious.

To address this and other limitations of alternative approaches, the present disclosure enables following training protocols while immersed in a virtual or augmented reality environment. According to various embodiments, content such as videos, movies, or 3D objects are displayed to a patient. The movement of this content in the space around the patient is used to guide the motions and activities defined by the protocol. This level of immersion encourages better adherence than watching a stationary screen.

An aspect of various physical therapies is the process of sway assessment. Conventional approaches to sway assessment are limited by the need for an approachable measurement device, the need to measure change in center of mass via the change of weight on feet using a platter, and inability to change scenery.

To address these and other limitations of conventional approaches, the present disclosure provides for measurement of sway in virtual or augmented reality. In particular, the present disclosure provide for calculating sway based on sensor feedback from handheld (or otherwise hand-affixed) sensors and from head mounted sensors. Using this sensor input, a test is provided that changes scenery in order to manipulate the visual & vestibular systems in order to get a comprehensive result.

Postural sway, in terms of human sense of balance, refers to horizontal movement around the center of mass. Sway can be a part of various test protocols, including: Fall risk; Athletic single leg stability; Limits of stability; or Postural stability.

Measurements of postural sway can provide accurate fall risk assessment and conditioning for adults, and neuromuscular control assessment, by quantifying the ability to maintain static or dynamic bilateral and unilateral postural stability on a static or dynamic surface.

Various clinical tests for balance may quantify balance in terms of various indices. A stability index may measure the average position from center. This measure does not indicate how much sway occurred during the test, but rather the position alone. A sway index may measure the standard deviation of the stability index over time. The higher the sway index, the more unsteady a subject was during the test. This provides an objective quantification of sway. For example, a pass/fail result of a test may be determined based on the sway index over a predetermined time period, such as 30 seconds. Likewise, a scale may be applied to the sway index, for example a value of 1 to 4 to characterize the sway where 1 corresponds to minimal sway, 4 corresponds to a fall.

Various advantages of using virtual or augmented reality as set out herein for assessing postural sway will be apparent. For example, center of mass assessment is improved over conventional approaches that rely on measuring the changes of weight on feet on a single platter. The actual average center of mass of a standing human being is generally at the Sacrum-2 point. This more precise center of mass point can be assessed and measured continuously using hand sensors and a head mounted display sensor in accordance with the present disclosure. These data are evaluated against posture guidelines provided in the VR/AR environment to provide a continuous index for center of mass. As set out below, such a continuous index may be generated at a rate of up to about 150 Hz. In some embodiments, data are collected and processed via inverse kinematics. In this way, the maximum range of motion for each tracked body part is recorded. A map of max range of motion may then be produced on a per-user basis.

In various embodiments, a patient's balance may be challenged through a change of scenery or environment. This allows better control over a user input than conventional approaches that rely on separately limiting visual, vestibular, and somatosensory feedback. For example, eyes may be closed to neutralize vision. A subject may stand on high density foam cushion to neutralize the somatosensory system. A subject may be placed in a visual conflict dome in order to neutralize the vestibular system.

In various embodiments, the systems of the present disclosure may present a predetermined rehabilitation protocol to one or more users. In various embodiments, the system may determine compliance with the predetermined rehabilitation protocol, e.g., by comparing recorded positional information from the one or more users to a set of positional data representing an ideal and/or standard procedure. In various embodiments, the compliance metric may be determined at the remote server. In various embodiments, the compliance metric may be determined as a measurement of how accurately and/or completely a user is performing a prescribed set of motions for the predetermined protocol. In various embodiments, the positional data of the user may be compared to positional data representative of the correct motions in the protocol. In various embodiments, the compliance metric may include a range of acceptable values. In various embodiments, the compliance metric may include a biometric measurement.

In various embodiments, the biometric measurement is selected from: heart rate, blood pressure, breathing rate, electrical activity of the muscles, electrical activity of the brain, pupil dilation, and perspiration.

In various embodiments, whether the biometric measurement is above a threshold is determined. When the biometric measurement is above the threshold, an additional adjustment to the training protocol is determined. The additional adjustment is applied to the training protocol until the biometric measurement is below the threshold. In various embodiments, the threshold is a target heart rate. In various embodiments, whether the biometric measurement is below a bottom threshold is determined. In various embodiments, an additional adjustment to the training protocol is determined when the biometric measurement is below the bottom threshold. The additional adjustment is applied to the training protocol until the biometric measurement is above the bottom threshold. In various embodiments, motion data and/or biometric measurements are logged in the electronic health record.

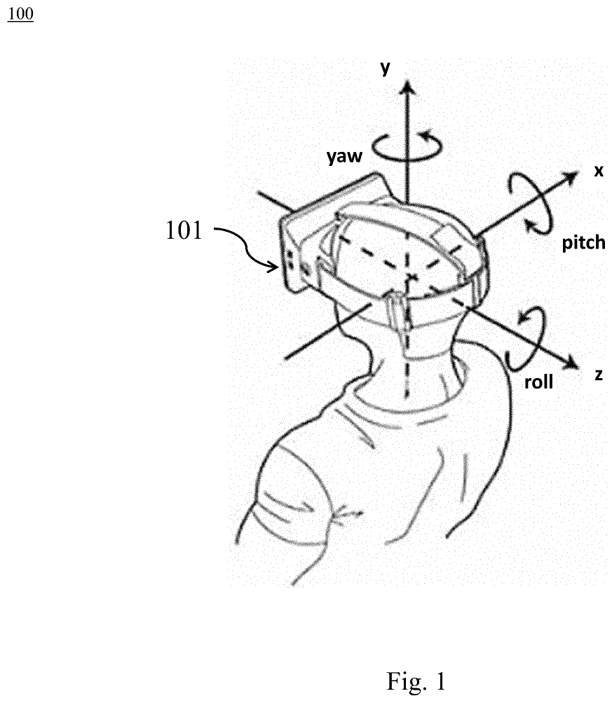

With reference now to FIG. 1, an exemplary virtual reality headset is illustrated according to embodiments of the present disclosure. In various embodiments, system 100 is used to collected data from motion sensors including hand sensors (not pictured), sensors included in headset 101, and additional sensors such as torso sensors or a stereo camera. In some embodiments, data from these sensors is collected at a rate of up to about 150 Hz. As pictured, data may be collected in six degrees of freedom: X—left/right; Y—up/down/height; Z—foreword/backward; P—pitch; R—roll; Y—yaw.

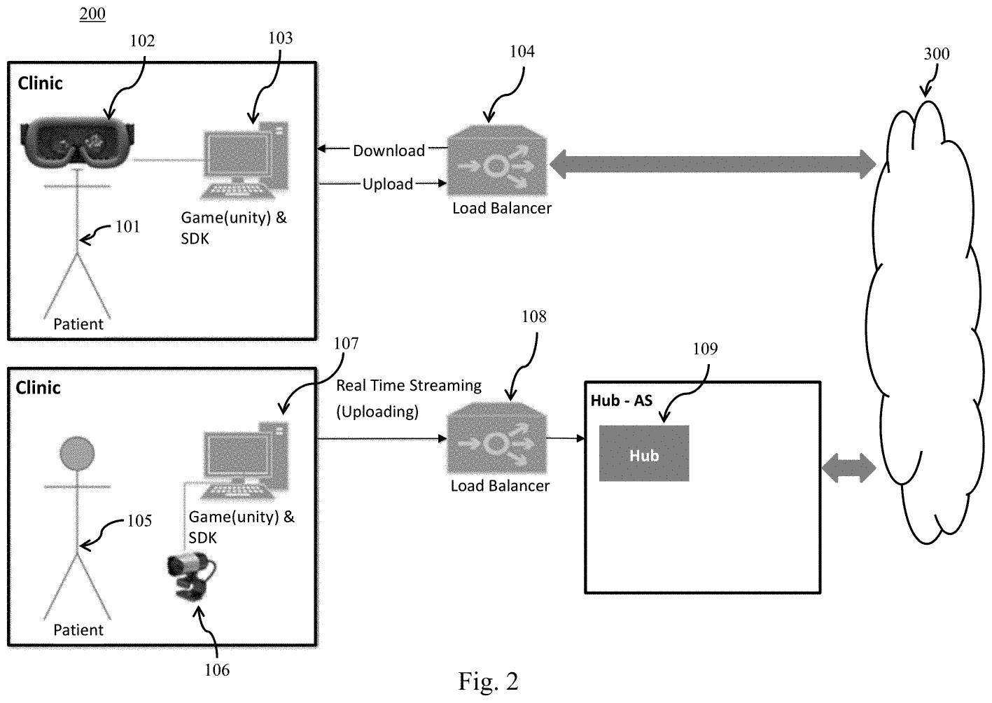

Referring to FIG. 2, an exemplary system according to embodiments of the present disclosure is illustrated. The collected data from the sensors can be stored on a database 304 for medical analysis in the exemplary architecture illustrated in FIG. 2. Data is gathered from user 101 by wearable 102. In some embodiments, computing node 103 is connected to wearable 102 by wired or wireless connection. In some embodiments, computing node 103 is integrated in wearable 102. In some embodiments, a load balancer 104 receives data from computing node 103 via a network, and divides the data among multiple cloud resources 300.

In some embodiments, camera 106 observes user 105. Video is provided to computing node 107, which in turn sends the video data via a network. In some embodiments, load balancer 108 receives data from computing node 107 via a network, and divides the data among multiple cloud resources 300. In some embodiments, hub 109 receives data from computing node 107 and stores or relays incoming video and event information for further processing.

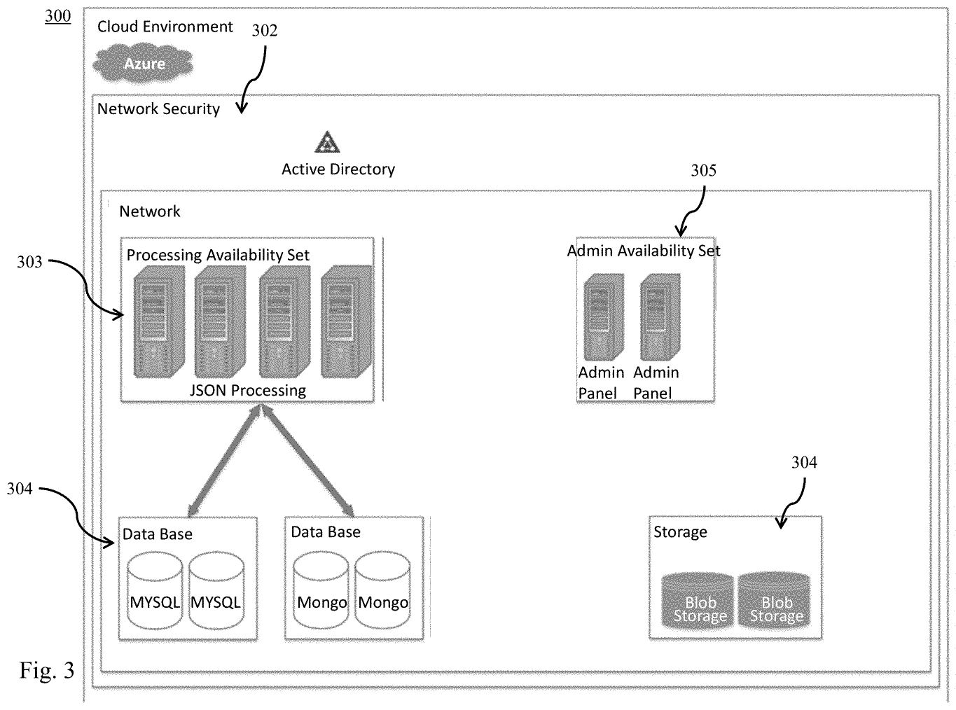

Referring to FIG. 3, an exemplary cloud environment according to embodiments of the present disclosure is illustrated. Various cloud platforms are suitable for use according to the present disclosure. A network security layer 302 applies security policy and rules with respect to service access. In some embodiments, Active Directory or equivalent directory services may be used for user authentication.

A set of processing servers 303 are responsible for receiving and analyzing data from the various user devices described herein. In various embodiments, processing servers 303 are also responsible for sending data, such as history information, to users upon request. The number of processing servers may be scaled to provide a desired level of redundancy and performance.

Processing servers 303 are connected to datastores 304. Datastores 304 may include multiple database types. For example, a SQL database such as MySQL may be used to maintain patient or doctor details, or user credentials. A NoSQL database such as MongoDB may be used to store large data files. Datastores 304 may be backed by storage 305.

In some embodiments, admin servers 306 provide a remotely accessible user interface, such as a web interface, for administering users and data of the system. The number of admin servers may be scaled to provide a desired level of redundancy and performance.

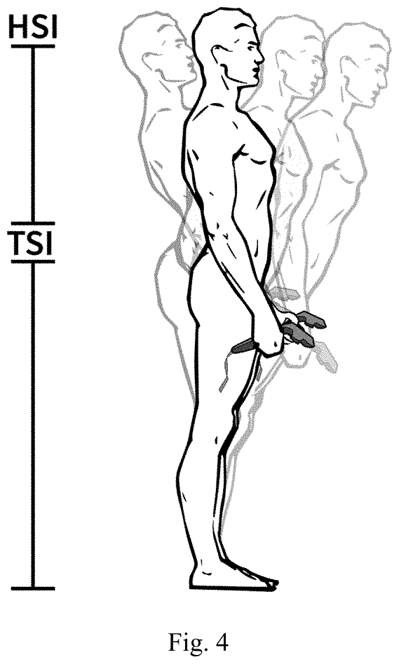

Referring now to FIG. 4, the Torso Sway Index (TSI) and Head Sway Index (HSI) of a person are illustrated. As set out herein, these indices, alone or in combination provide improves assessment of fall risk and postural stability, both static and dynamic.

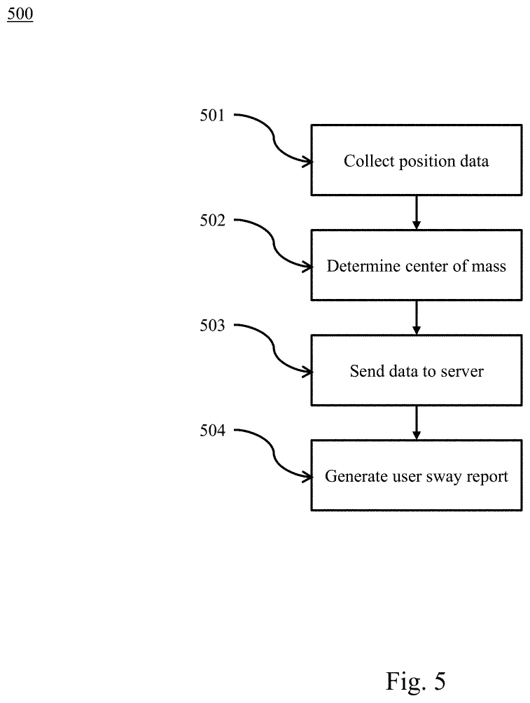

Referring to FIG. 5, a method of sway assessment according to embodiments of the present disclosure is illustrated. At 501, position data is collected from a user. In some embodiments, the position data is collected from sensors including those within a head mounted display or handheld controllers. In some embodiments, data is collected at a rate of up to about 150 Hz. In some embodiments, a user is provided with per-assessment guidance on which sensors are needed and in what positions (e.g., hand controllers above the waist). In some embodiments, a user is provided with guidance as to the precise postural position of the patient (e.g., tandem standing).

At 502, the positional data is processed to determine the center of mass of the user. In some embodiments, the center of mass is computed in three dimensions.

In some embodiments, the center of mass is represented by a 3-dimensional position calculated from the head mounted display and two hand sensors. This point, C, may be calculated as a weighted average of the three sensors according to Equation 1, where X, Y, Z are the coordinates of a given sensor, a, b, c are constants, rhs identified the left hand sensor, lhs identifies the right hand sensor, and hmd identifies the head-mounted display.

C=a(Xᵣₕₛ,Yᵣₕₛ,Zᵣₕₛ)+b(Xₗₕₛ,Yₗₕₛ,Zₗₕₛ)+c(X_{nmd},Y_{hmd},Z_{hmd}) Equation 1

In various embodiments, the constants a, b, c are determined based on individual attributes, including distance between hands and head, and distance between hands. In some embodiments, constants a, b, c are tuned by application of machine learning. In some embodiments, a, b, c are adjusted based on patient dimensions derived from stereo camera data.

In addition to the center of gravity, the head sway index (HSI) and torso sway index (TSI) may be computed and stored at regular intervals. The head sway index is computed from X_{hmd}, Y_{hmd}, Z_{hmd}, representing the coordinates of the head-mounted display. The torso sway index is computed from Xᵣₕₛ, Yᵣₕₛ, Zᵣₕₛ, and Xₗₕₛ, Yₗₕₛ, Zₗₕₛ, representing the coordinates of the extremities.

At 503, the raw position data and center of mass are sent to a remote server. At the server, additional analysis may be conducted. In some embodiments, a sway index is computed.

At 504, a report of user sway is generated based on the center of mass over time. In some embodiments, the report is sent to the user via a network.

In this way, systems according to the present disclosure are continuously calculating the patient's center of mass using a smart algorithm and giving the patient instruction in a VR environment about his posture during the test. The center of mass of the patient is saved at up to 150 Hz on a server, enabling the calculation of different sway indexes (e.g., sway index or stability index). A 3-dimensional dynamic result of the patient's center of mass is provided, located on average in the S2 vertebra point while standing.

A patient's balance may be challenged through a change of scenery or environment. The challenge within the VR/AR environment may include a challenge to the visual and vestibular systems in order to get a more complex and comprehensive test. For example, the vestibular system may be manipulated by changing the virtual/augmented experience by slowly rotating the horizon to effect balance. In another example, the vision system may be manipulated by changing the virtual/augmented experience by changing the light in the environment to make it harder to notice details. In another example, scenery may be adjusted during the test according to the patient sway index in real time. This enables a more precise comprehensive result regarding a patient's postural sway status.

In various embodiments, sway may be measured during different tasks. Using VR/AR allows testing of a patient's sway in different tasks and scenarios, from day to day functional scenarios to specific scenarios crafted for the sway test.

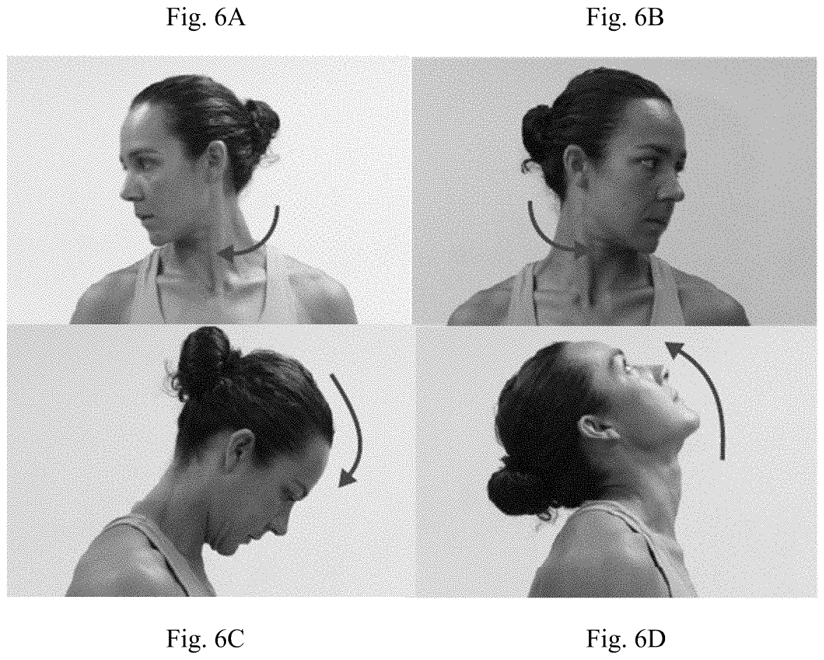

Referring now to FIGS. 6A-6D, various exemplary motions of a user's neck are illustrated. In particular, FIGS. 6A-6D illustrate various neck movement exercises that may be utilized in various embodiments of the systems described herein. The user may be instructed to sit in the correct position before performing any of the below exercises. To facilitate these motions, in various embodiments, a moving 2D or 3D object is displayed through a VR or AR device to the user. This object moves around the user's space, guiding the performance of specific physical training protocols. The user, in order to follow the object and succeed in the training, must physically do the desired motions by following the object's movement in space. It will be appreciated that although the present example is given in terms of neck motions, tracking of the virtual object may be based on the motion of different body parts, depending on the training protocol performed. For example, a handheld sensor may be tracked, and the user prompted to move their arm to remain pointing at a virtual object.

FIGS. 6A-6B illustrate neck rotation where the user may be instructed to gently turn their head from one side to the other. The user may be instructed to progressively aim their head so that they see the wall in line with their shoulder.

FIGS. 6C-6D illustrate neck bending and extension where the user may be instructed to gently bend their head towards their chest. The user may be instructed to lead the movement with their chin and, moving the chin first, to bring their head back to the upright position and gently roll it back to look up towards the ceiling. The user may be instructed to, leading with their chin, return their head to the upright position. Any of the above exercises may be performed a predetermined number of times, e.g., ten times.

In various embodiments, training protocols are based on standard rehabilitation exercises. For example, additional neck movements suitable for neck rehabilitation using various embodiments of the systems described herein may be found in Guidelines for the management of acute whiplash associated disorders for health professionals, 3^{rd} Edition, 2014, available at https://www.sira.nsw.gov.au/resources-library/motor-accident-resources/publications/for-professionals/whiplash-resources/SIRA08104-Whiplash-Guidelines-1117-396479.pdf, which is hereby incorporated by reference. However, it will be appreciated that the versatility of the virtual environment enables a range of exercises that are not practical when relying on physical cues.

In an exemplary neck physical training protocol, a 2D or 3D object moves in the space around the user. The user is directed to follow the object with their gaze, thus moving their neck in the direction the object moves, performing the neck movements suitable for neck rehabilitation.

In an exemplary arm/shoulder/back rehabilitation protocol, a 2D or 3D object moves in the space around the user. The user is directed to follow the object with their arm position, thus moving their arm in the direction the object moves.

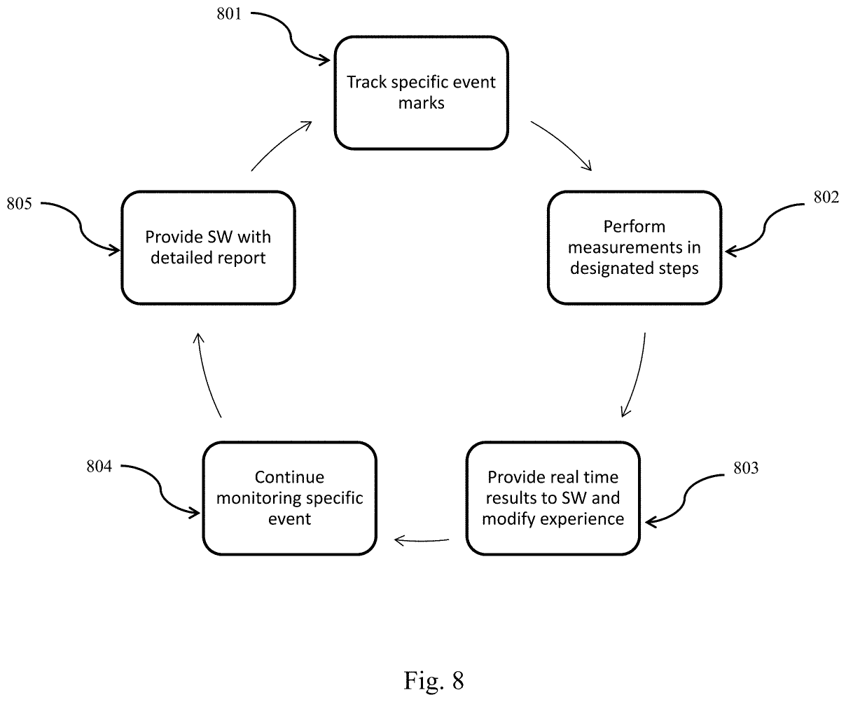

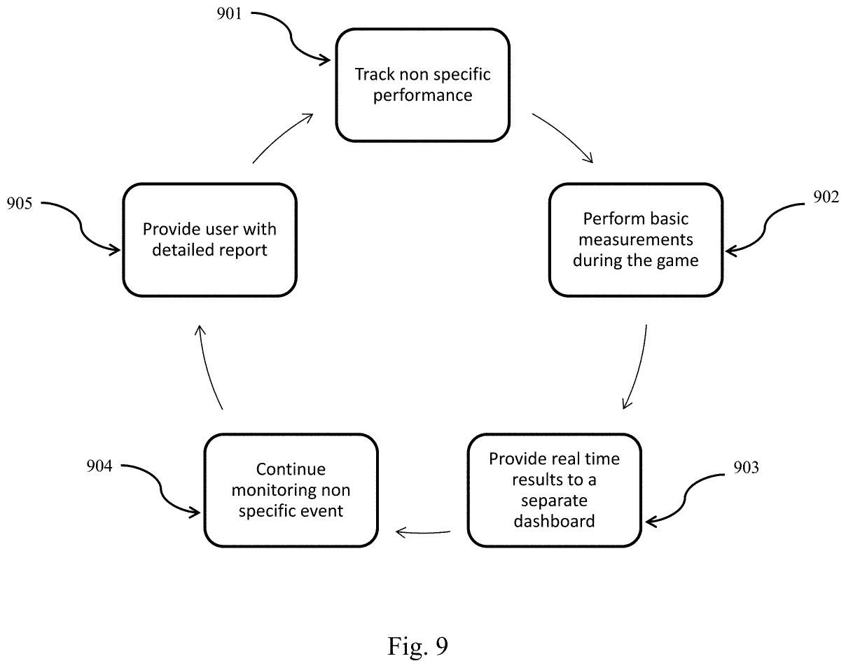

Referring to FIGS. 8-9, processes for monitoring and assessing performance in virtual or augmented reality are illustrated. As described above, in various embodiments, a modular system is provided that can interface with third party augmented or virtual reality systems. In this way, an additional layer of data may be provided beyond what is otherwise present in an immersive environment. In particular, algorithms may be run in the background of any immersive computing experience to monitor and assess real time motor, cognitive, and mental actions taken by the user in the environment, providing this data to both users and developers to enhance and modify the experience.

By collecting data in VR/AR, the user is constantly observed as if he or she was in a checkup room, regardless of the particular experience the user is engaged with. Referring to FIG. 8, in some embodiments, an SDK is provided to third party application developers. In such embodiments, the data provided can help modify and improve user experience in real time. For example, at 801, specific event markers are tracked within the VR or AR experience. At 802, measurement of the user is performed at each step. At 803, real time results are provided to the containing software. In some embodiments, this is provided through an event listener interface, although it will be appreciated that various approaches are available for providing data from a modular system such as described herein to a containing software application. The containing software may then modify the VR/AR experience according to the data. At 804, specific events are monitored on an ongoing basis, for example, changes in motion by the user. In various embodiments, the event marker may be a specific, marked location in the VR/AR environment. At 805, a detailed report is provided to the containing software. For example, a detailed report may be provided at the conclusion of a gaming session.

In various embodiments, the detailed report may include a compliance metric. In various embodiments, the compliance metric may be determined from positional information of the user collected as the user performs an activity. In various embodiments, the user may be instructed to make a motion with a particular one or more body parts (e.g., head, neck, one or both arms, one or both legs, one or both feet, one or both hands, etc.) towards the event marker. In various embodiments, based on a specific rehabilitation, the user may be instructed to repeat the activity, such as, for example, the motion towards the event marker.

In various embodiments, the event marker may change locations in the user's field of view in the VR/AR environment after a predetermined number of repetitions and/or a predetermined compliance metric is met. In various embodiments, the event marker may change locations within the user's field of view to a location that increases the difficulty of the activity. For example, in a rehabilitation setting, after completing a predetermined number of repetitions of an activity successfully (e.g., a shoulder range-of-motion activity), the VR/AR system may, for example, increase the range of motion required by the activity to increase the difficulty and/or increase the number of repetitions. In various embodiments, the VR/AR system may automatically increase the difficulty of the activity on a predetermined schedule (e.g., daily, weekly, every other rehabilitation session, etc.).

In various embodiments, the detailed report may be saved to an electronic health record. In various embodiments, the detailed report may be shared with a health care provider and/or a third party involved in the rehabilitation of the patient (e.g., insurance company, pharmacy, etc.).

Referring to FIG. 9, a loosely coupled approach is adopted in various embodiments, in which monitoring is performed in parallel to a VR or AR experience without interfacing directly with the game or other VR software. In such embodiments, data are saved by the platform to track user progress and provide the user with valuable analytics on his or her progress—e.g., it can provide him or her the number of calories burned in virtual reality. In particular, at 901, general performance is tracked. At 902, general measurements of the user are performed, for example, during a game. In this embodiments, measurements are conducted on an ongoing basis without the benefit of direct connectivity to the host software, as would be available in the embedded scenario discussed with regard to FIG. 8. At 903, real time results are provided to a dashboard. In this embodiment, the dashboard may be separate from the game experience, for example on a supplemental display. At 904, monitoring is continued. At 905, a detailed report is provided to the user.

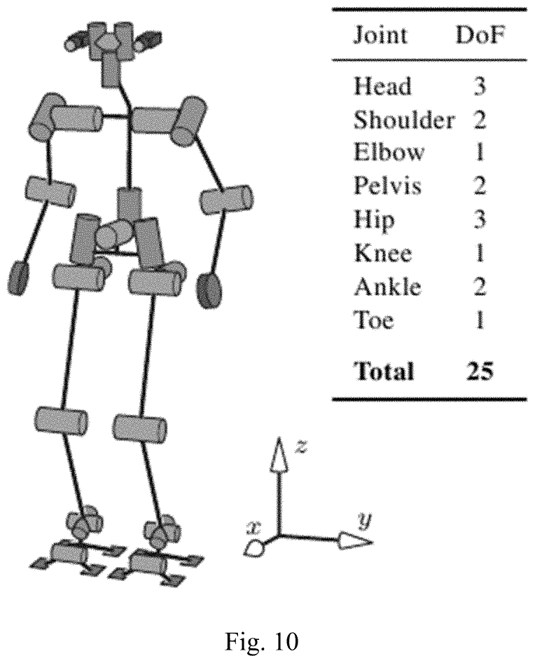

Referring to FIG. 10, the degrees of freedom on various joints in an exemplary human kinematic model are illustrated. It will be appreciated that as described above, the range of motion may be tracked for each of the various joints in accordance with the present disclosure.

Monitoring/Assessing Proprioception

In various embodiments, the systems and methods described herein may be used to monitor and/or assess proprioception of a user. Proprioception is the sense of position of one's own body parts in space. It can be damaged in various pathologies and affect a patient's ability to produce functional movements, which can result in decreased functionality in everyday living actions. For that reason, practicing and improving proprioception is vital to succeeding in the process of rehabilitation.

Practicing proprioception may be done with manual methods, which aim to facilitate mechanoreceptors that are a crucial for the proprioception abilities and require performing controlled movement with the relevant body part. For example, when practicing shoulder proprioception, the patient can be asked to roll a foam roller on a wall in front of him with his upper extremity, aiming at targets that are located in different locations on the wall.

Additional methods include practicing and evaluating proprioception with real time visual feedback, which aims to activate motor control learning processes, thus improving the sensorimotor system. For example, for neck proprioception practice and evaluation, a Tracker laser kit system uses a laser pointer, which is put on the patient's head, and a target that is located on a wall in front of the patient, with drawn circles and lines. This method enables the physician to evaluate the patient's Joint Position Error (JPE), following the lines performing neck movements according to a clinician's guidelines enables practicing neck proprioception.

Performing proprioception assessment and practice without additional accessories does not give any concrete information on patient's proprioception abilities, and progression cannot be seen over time. Systems such as the Tracker laser are cumbersome and hard to operate; to get information on a patient's proprioception abilities, one needs to measure the distances between the required positions and the performed position in space. Additionally, in today's known solutions, the evaluation and practice process can be boring for the patient and requires clinician's guidelines and supervision.

Quantifying proprioception abilities easily provides the clinician an “Asterix,” which is an objective value that can give clinical information about a patient and reflect whether the treatment is helping the patient or not.

In addition, the whole rehabilitation experience today can be boring and exhausting, both in clinic and at home. Consequently, patients may not do their prescribed home exercise, which makes the patient's recovery difficult. Current solutions also often lack the ability to adjust the training in tele-rehabilitation, which can benefit clinicians and patients in the rehabilitation process.

Various embodiments disclosed herein relate to using VR/AR to evaluate and practice proprioception. Proprioception rehabilitation principles can be combined and immersed in VR/AR abilities as follows:

a. High tracking quality—Proprioception allows the formation of a mental model, describing the spatial and relational dispositional of the body and its parts. A virtual reality system overlays the normal proprioceptive data that is used to form a mental model of the body with sensory data that is supplied by the computer-generated displays. For an effective virtual reality, the proprioceptive information and sensory feedback should be consistent. This is done by the correct capturing of the movement of the user, and simulating it in the virtual environment, in order to increase a sense of immersion. Hardware such as, e.g., Oculus Rift and HTC Vive allows tracking samples in a very high rate per second, with position accuracy of under 1 millimeter, and rotation precision of 0.1 degrees and under, according to manufacturer's statement.

b. Live feedback—Non-proprioceptive feedback may be used to improve proprioceptive function. For example, active proprioceptive training in the form of target reaching assisted with acoustic feedback reduces target reaching error immediately after training. However, when subjects have to reach to remembered targets from prior training sessions (e.g., approximately 2 days prior), the efficiency of reaching reduces by approximately 25%. Further evaluation shows that this reduction in target reaching efficiency occurs mainly due to the inaccurate internal representation of the space rather than inaccurate motor planning. This conclusion is based on the training of one hand to reach proprioceptive targets and testing the other hand for accuracy in reach position. Further, passive or active movement training shows that the presence of feedback may affect sensorimotor function. When no feedback is given, there is no significant differences of corticospinal excitability before and after passive wrist movement, or between passive and active training groups. With visual feedback, active training is shown to be superior to passive training. A significant improvement in spatial accuracy of an active wrist tracking test (with feedback) is shown following training with an active tracking task versus a group performing passive wrist tracking that included online visual feedback and fixed auditory feedback. Thus, active training in the presence of visual feedback shows significant improvements in proprioceptive acuity in healthy subjects.

c. Quantify proprioception—providing patients and clinicians with tools that can quantify proprioception abilities easily provides clinician and patient an “Asterix,” which is an objective value that can give clinical information about patient's performance and reflect whether the treatment is beneficial. Few known solutions provide the ability to quantify proprioception; those solutions are clumsy to use and require the clinician to measure distances with a ruler.

d. Relevant sensory activation—Proprioception is the ability to sense the position of the muscles, and the relative position among contiguous body parts. Using VR/AR, the sight is blocked when the patient wears the virtual reality glasses, so they are unable to see themselves moving their upper trunk. This hardens some tasks such as motion coordination, automatic body responses and awareness of self-position across the space. As a result, extra effort must be done by other sensors, which may accelerate the treatment and increase its effectiveness.

e. Gamifying the rehabilitation process—using a VR/AR system transforms the proprioception evaluation and practice from a boring and repetitive training into a fun game by designing the virtual environment. Consequently, the player is focused on the game and its performance, creating external que focus on the proprioceptive training, which has positive clinical influence.

f. Tele-rehabilitation—Ability to adjust and perform proprioceptive training in tele-rehabilitation or while not having clinician supervising the patient compared to obligation of the clinician to be next to the patient when training is performed in order to guide and supervise the patient.

In some embodiments, VR/AR is used to enable proprioception assessment and practice through the following steps:

1. Create VR/AR experiences that will make patients perform fine motor-controlled movements and enable sensory motor control assessment and workout. The experiences will be adjusted according to the patient's needs by a control panel with changeable parameters for proprioception workout.

2. Positional data from wearable sensors is tracked and collected at high sample rate. Each assessment/practice will include guidance of which sensors are needed and in what position in space, making the patient perform the relevant movement defined by the clinician. This will result in different joint positions performed by the patient and precise position in space tracking of relevant body part produced by the patient (e.g. neck 30 degrees right rotation).

3. Raw and calculated data are sent to the server, where they are logged, and additional analysis is performed.

4. Results are sent from server via SDK to the patient with a final report.

The following is an example of such procedure done relying on joint position sense (JPS) principles using VR/AR:

a. Patient will be guided to perform a movement with the relevant body part to a specific point in space chosen by the clinician and memorize this point.

b. Patient returns to neutral position with the relevant body part and instructed to produce and reach the required point in space, with eyes closed or with a clean objectless environment presented in head mounted display (HMD). This action will rely on proprioceptive elements.

c. The required and performed points in space are recoded and the difference between them represents the joint position error (JPE).

d. Analysis of the results will be made, enabling to quantify proprioception abilities and reflect clinical information on the patient.

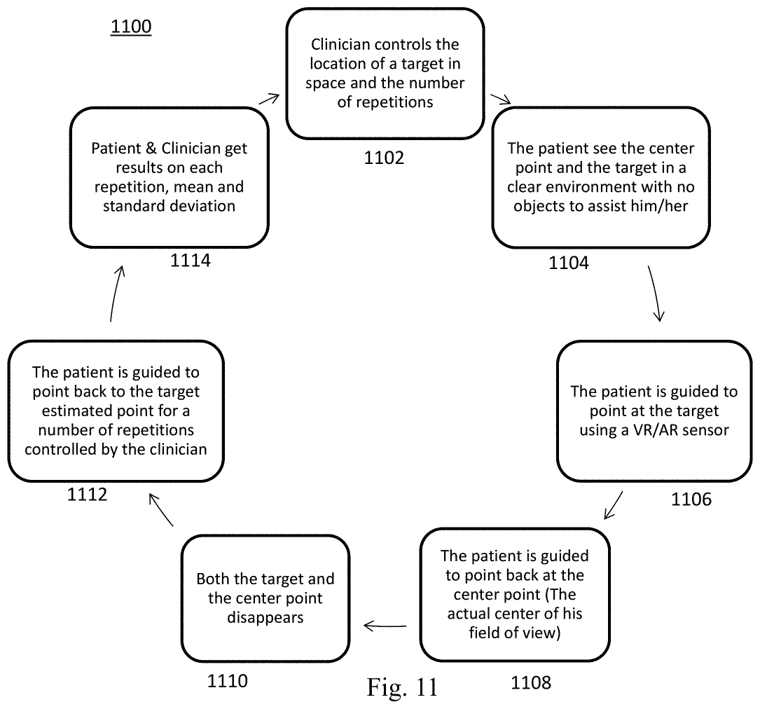

FIG. 11 is a flow diagram illustrating an exemplary process 1100 for neck proprioception rehabilitation. At 1102, a clinician controls the location of a target in space and the number of repetitions for a patient. At 1104, the patient sees the center point and the target in a clear environment with no objects to assist the patient. At 1106, the patient is guided to point at the target using a VR/AR sensor. At 1108, the patient is guided to point back at the center point (The actual center of his field of view). At 1110, both the target and the center point disappears. At 1112, the patient is guided to point back to the target estimated point for a number of repetitions controlled by the clinician. At 1114, the patient and the clinician receive results on each repetition, in addition to statistics, such as, e.g., mean and standard deviation. In various embodiments, the process may repeat back to 1102 for any suitable number of repetitions.

In this procedure aimed to train and measure Joint Positional Awareness (JPA) 1201, the player is required to look at a target, look back at the center point, and then try to recreate to the same point in space the target appeared after the entire view is hidden, activating neck proprioceptive mechanoreceptors.

Clinicians can perform an adjustment according to the patient's needs, and control the number of repetitions for this procedure, and the locations of the required point is space the patient is supposed to recreate. In various embodiments, the clinician may instruct the patient to perform a first activity, e.g., look at (or move a body part towards) a first location and then recreate the same motion with the visual field restricted or blacked out (in part or in total). In various embodiments, positional information of the user may be recorded during this process. In various embodiments, the positional information of the patient while performing the activity may be compared against a predetermined set of positional information representing an ideal path to the target. In various embodiments, the systems of the present disclosure may determine a compliance metric as described in more detail above based on, for example, how closely a patient recreates the initial motion while having their visual field restricted or blacked out. In various embodiments, the compliance metric may be a score. In various embodiments, the compliance metric may be recorded in an electronic health record. In various embodiments, the compliance metric may be presented to the user (e.g., visually, audibly, etc.). Based on the patient's performance of completing this activity, the clinician may instruct the patient to perform a second activity, e.g., look at (or move a body part towards) a second location and then recreate the same motion with the visual field restricted or blacked out (in part or in total). In various embodiments, a compliance metric may also be determined for the second activity.

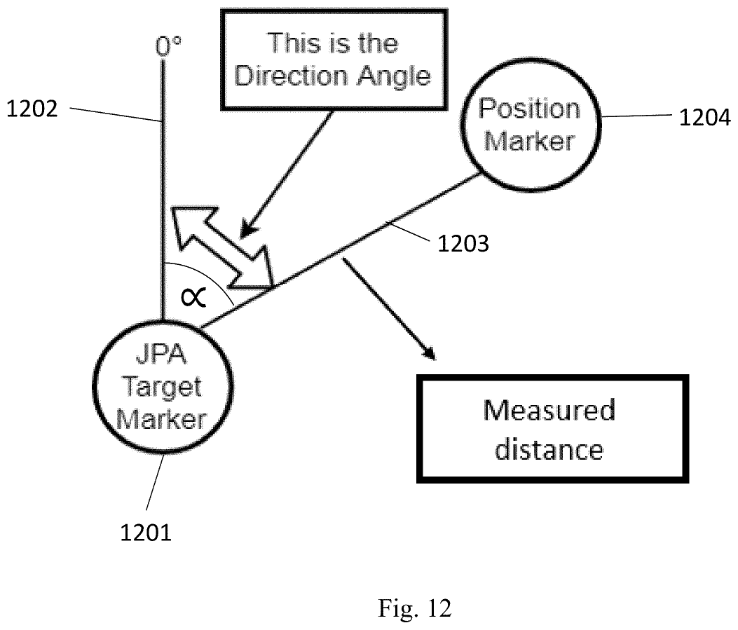

The distance between the required position and the actual position in space the patient was supposed to recreate may be measured by distance and direction and are pointing on patient's JPS as illustrated in FIG. 12. The angle between a reference axis 1202 and a vector 1203 pointing towards a position marker 1204 may be measure in Euler angles. In various embodiments, for example, in a 2D plane, the angle α represents the angle the patient is to move. In various embodiments, the vector 1203 includes a linear distance the patient is to move.

The average between the results according to the number of repetitions and the locations in space that were chosen is calculated and presented at the end of the procedure, presenting an “Asterix” that enables to see progression over time.

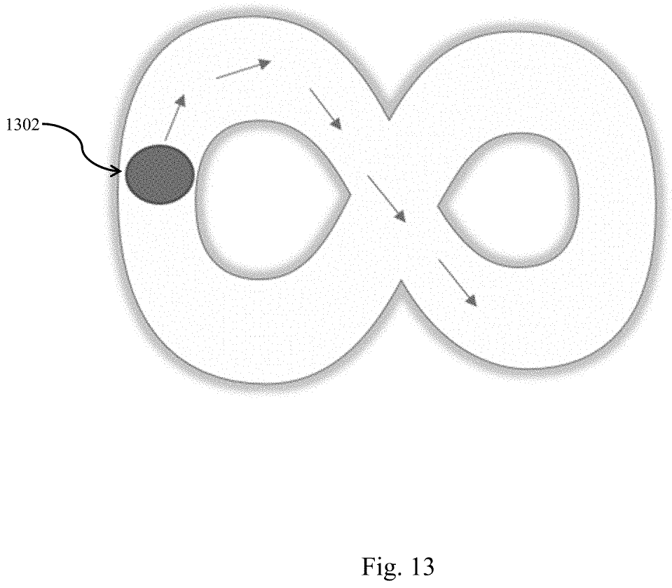

FIG. 13 illustrates another example of a procedure that enables practicing and assessing proprioception. In this example, the patient performs a controlled neck movement, creating a trail in the virtual environment that can be shaped as an eight figure. The patient moves his neck and follow a point 1302 that moves inside the trail.

Tracking movements facilitate mechanoreceptors in the sensorimotor system, enabling to practice proprioception. This can be done for different body parts such as neck, upper and lower extremities.

The game can be adjusted by a clinician according to the patient's needs by the following parameters:

a. Direction—figure eight can be horizontal or vertical or both

b. Size—how big is the figure eight, enables to control patient required range of motion to reach.

c. Track width—influences the size of the target, adding another layer of difficulty level. The smaller the target, the harder it will be to follow it.

d. Target speed—control the moving target's speed that the patient is required to follow.

e. Location in space—control path's position in space allowing to facilitate relevant proprioceptive mechanoreceptors.

f. Number of repetitions—adding another layer of difficulty, effecting on the training content and duration.

The system's high sample rate enables the collection of qualitative information on the patient's performance, analyzing it and presenting at the end of the procedure:

a. Accuracy index will be calculated by the following rules:
i. The player's looking angle is compared to the angle of the Target, relative to the game's zero point.
   1. The angle between the player's looking angle and the target's angle is called the Delta Angle and is calculated for every sample taken.

b. Path deviation index will be calculated by the following rules:
i. If the player's gaze leaves the track and touches the Path Border, a deviation is detected.
ii. A new deviation will not be detected until the player's gaze returns to the track.
iii. If the player's gaze is not on the Moving Target, but still inside the track, it is not considered a deviation.
iv. The application will count the number of such deviations and display the number to the user in the end of the procedure.

These parameters presented at the end of the procedure, presenting an “Asterix” that enables to see progression over time, giving clinical information about the patient's performance.

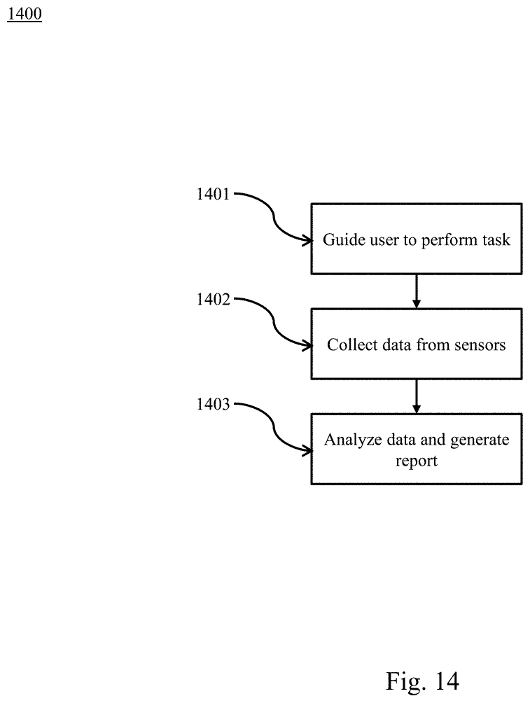

Referring now to FIG. 14, a method of for assessing and practicing proprioception in virtual or augmented reality environments are disclosed. At 1401, a user is guided to perform a task involving movement of a given body part of the user via a virtual or augmented reality display. At 1402, data is collected from a plurality of sensors relating to the user's performance of the task. At 1403, the data is analyzed and a report is generated reflecting the proprioception abilities of the user based on the performance of the task.

Closed Circuit Assessment, Decision-Making, and Protocol Rendering

In various embodiments, systems for, computer program products for, and method of closed circuit assessment, decision-making, and protocol rendering in virtual reality (VR) or augmented reality (AR) environments are provided.

The connection between patient assessment and screening to a clinical decision-making and treatment protocol is currently subject to a clinician's discretion and can lack consistency between sessions and between clinicians. It is difficult to monitor this entire process, and it is often lacking in documentation. Most existing solutions are one dimensional and do not allow an automated close circuit system.

The VR/AR technology according to various embodiments provides a fully immersive environment that enables a user to be immersed in an automated close circuit system. Within this environment, a virtual clinician (an avatar) utilizing machine learning and artificial intelligence (AI) can communicate with, assess, and monitor the patient and create an automated close circuit decision to identify the right treatment protocol for the user. The VR/AR technology also allows the environment to be manipulated, e.g., multiple layers can be added to the environment to create different tasks and situations for the users. This enables determining a more precise evaluation, training, or treatment regimen, while monitoring the user constantly and providing immediate feedback. This integrated VR platform enables costs to be reduced and objectivity and accessibility improved for highly accurate measurements and fully detailed outputs. The solution is a portable and accessible tool that can be used both in local facilities or remote access.

Currently, initial screening is done by clinicians via questionnaires. In various embodiments, the initial screening in done by an avatar using AI or machine learning. The avatar reacts to the patient responses though predetermined and real-time algorithms to determine the best treatment protocol that is suitable for each specific patient.

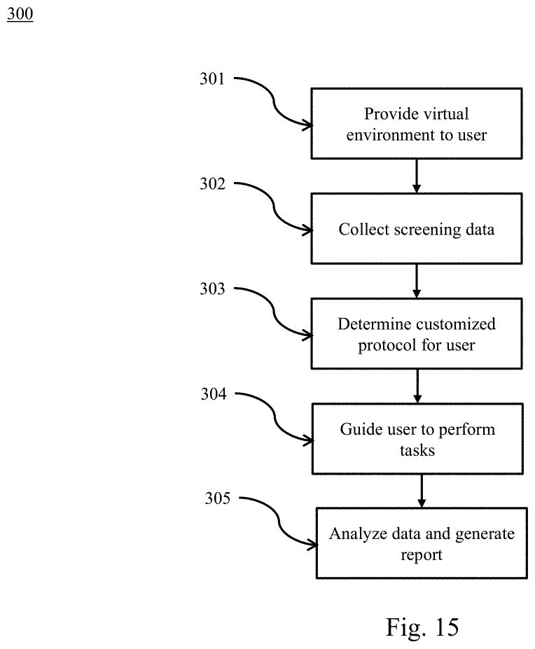

Referring now to FIG. 15, a method 1500 of for closed circuit assessment, decision-making, and protocol rendering in a virtual or augmented reality environments is disclosed. At 1501, a virtual environment is provided to the user via a virtual or augmented reality system. The virtual environment includes an avatar using machine learning or artificial intelligence to communicate with the user. At 1502, screening data is collected from the user's interaction with the avatar in the virtual environment. At 1503, a customized evaluation, training, or treatment protocol is determined for the user based at least in part on the screening data. At 1504, the user is guided to perform a task in the evaluation, training, or treatment protocol via the virtual or augmented reality system. Data is collected from a plurality of sensors relating to the user's performance of the task. At 1505, the collected data is analyzed and a report is generated based on the user's performance of the task.

In various embodiments, screening data is evaluated for abnormal data. For example, a machine learning system may be trained to identify outliers in biometric data. In this way, a user may be notified if there is a significant variation in their data. The user may be advised to see a clinician if there is a significant change in screening and/or performance data. In some embodiments, the avatar provide such advice to the user.

The following is a non-limiting example of the use of a closed circuit assessment, decision-making, and protocol rendering in accordance with various embodiments. A user wears a VR/AR headset and enters a virtual environment. The user is greeted by an Avatar using AI and machine learning to perform specific screenings that are customized to the current state and specific characteristics of the user. After analyzing the user's responses and taking into account the history and performance of the user and the user's data, the VR environment is adjusted in order to create the most suitable treatment protocol for the user. During the treatment/workout session, the avatar constantly monitors and provides feedback for the user and continues to adjust the VR environment constantly. At the end of the session the Avatar can perform additional screening, provide feedback to the user, and recommend the next step that is most suitable for the user. After each session the user will be able to access all his or her data and performance evaluations.

A Picture Archiving and Communication System (PACS) is a medical imaging system that provides storage and access to images from multiple modalities. In many healthcare environments, electronic images and reports are transmitted digitally via PACS, thus eliminating the need to manually file, retrieve, or transport film jackets. A standard format for PACS image storage and transfer is DICOM (Digital Imaging and Communications in Medicine). Non-image data, such as scanned documents, may be incorporated using various standard formats such as PDF (Portable Document Format) encapsulated in DICOM.

An electronic health record (EHR), or electronic medical record (EMR), may refer to the systematized collection of patient and population electronically-stored health information in a digital format. These records can be shared across different health care settings and may extend beyond the information available in a PACS discussed above. Records may be shared through network-connected, enterprise-wide information systems or other information networks and exchanges. EHRs may include a range of data, including demographics, medical history, medication and allergies, immunization status, laboratory test results, radiology images, vital signs, personal statistics like age and weight, and billing information.

EHR systems may be designed to store data and capture the state of a patient across time. In this way, the need to track down a patient's previous paper medical records is eliminated. In addition, an EHR system may assist in ensuring that data is accurate and legible. It may reduce risk of data replication as the data is centralized. Due to the digital information being searchable, EMRs may be more effective when extracting medical data for the examination of possible trends and long term changes in a patient. Population-based studies of medical records may also be facilitated by the widespread adoption of EHRs and EMRs.

Health Level-7 or HL7 refers to a set of international standards for transfer of clinical and administrative data between software applications used by various healthcare providers. These standards focus on the application layer, which is layer 7 in the OSI model. Hospitals and other healthcare provider organizations may have many different computer systems used for everything from billing records to patient tracking. Ideally, all of these systems may communicate with each other when they receive new information or when they wish to retrieve information, but adoption of such approaches is not widespread. These data standards are meant to allow healthcare organizations to easily share clinical information. This ability to exchange information may help to minimize variability in medical care and the tendency for medical care to be geographically isolated.

In various systems, connections between a PACS, Electronic Medical Record (EMR), Hospital Information System (HIS), Radiology Information System (RIS), or report repository are provided. In this way, records and reports form the EMR may be ingested for analysis. For example, in addition to ingesting and storing HL7 orders and results messages, ADT messages may be used, or an EMR, RIS, or report repository may be queried directly via product specific mechanisms. Such mechanisms include Fast Health Interoperability Resources (FHIR) for relevant clinical information. Clinical data may also be obtained via receipt of various HL7 CDA documents such as a Continuity of Care Document (CCD). Various additional proprietary or site-customized query methods may also be employed in addition to the standard methods.

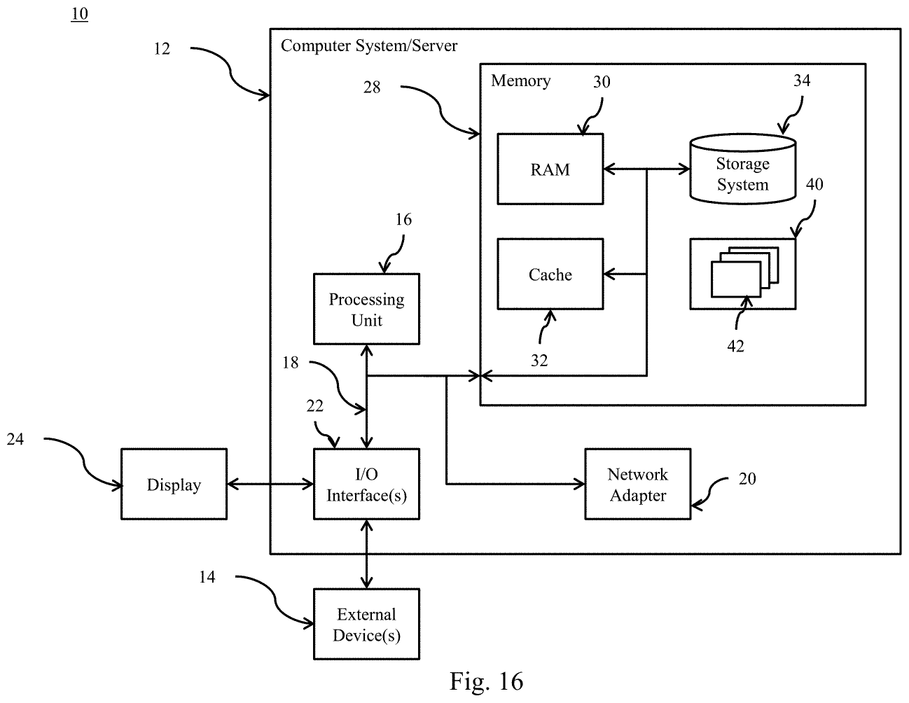

Referring now to FIG. 16, a schematic of an example of a computing node is shown. Computing node 10 is only one example of a suitable computing node and is not intended to suggest any limitation as to the scope of use or functionality of embodiments of the invention described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 16, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A method comprising:
providing a virtual environment to a user via a virtual or augmented reality system;
providing one or more event markers at a first location within the virtual or augmented reality environment;
adjusting the position of the one or more event markers to a second location within the virtual or augmented reality environment, the first location and the second location having a first distance therebetween;
collecting a first set of data based on the user's interaction with the one or more event markers, the first set of data comprising positional data of the user;
providing the first set of data to a remote server via a network;
determining a compliance metric based on the first set of data;
when the compliance metric differs from a predetermined range, applying a first adjustment to the one or more event markers.

2. The method of claim 1, wherein the event marker comprises a visual object displayed within the virtual or augmented reality environment.

3. The method of claim 1, further comprising adjusting the position of the event marker to a third location based on the applied first adjustment.

4. The method of claim 1, wherein the first adjustment comprises a speed of motion of the event marker as the position of the event marker is adjusted.

5. The method of claim 4, wherein the first adjustment comprises a slower speed.

6. The method of claim 4, wherein the first adjustment comprises a faster speed.

7. The method of claim 1, wherein the first adjustment comprises a change in distance of the event marker as the position of the event marker is adjusted.

8. The method of claim 7, wherein the first adjustment comprises a second distance that is greater than the first distance.

9. The method of claim 7, wherein the first adjustment comprises a second distance that is less than the first distance.

10. The method of claim 1, wherein the first adjustment comprises an increase in a number of repetitions of the user interaction with the event marker.

11. The method of claim 1, wherein the first adjustment comprises a decrease in a number of repetitions of the user interaction with the event marker.

12. A system comprising:
a computing node comprising a computer readable storage medium having program instructions embodied therewith, the program instructions executable by a processor of the computing node to cause the processor to perform a method comprising:
providing a virtual environment to a user via a virtual or augmented reality system;
providing one or more event markers at a first location within the virtual or augmented reality environment;
adjusting the position of the one or more event markers to a second location within the virtual or augmented reality environment;
collecting a first set of data based on the user's interaction with the one or more event markers, the first set of data comprising positional data of the user;
providing the first set of data to a remote server via a network;
determining a compliance metric based on the first set of data;
when the compliance metric differs from a predetermined range, applying an adjustment to the one or more event markers.

13-22. (canceled)

23. A computer program product for monitoring and assessing performance of a user, the computer program product comprising a computer readable storage medium having program instructions embodied therewith, the program instructions executable by a processor to cause the processor to perform a method comprising:
providing a virtual environment to a user via a virtual or augmented reality system;
providing one or more event markers at a first location within the virtual or augmented reality environment;
adjusting the position of the one or more event markers to a second location within the virtual or augmented reality environment, the first location and the second location having a first distance therebetween;
collecting a first set of data based on the user's interaction with the one or more event markers, the first set of data comprising positional data of the user;
providing the first set of data to a remote server via a network;
determining a compliance metric based on the first set of data;
when the compliance metric differs from a predetermined range, applying a first adjustment to the one or more event markers.

24. (canceled)

25. The computer program product of claim 23, further comprising adjusting the position of the event marker to a third location based on the applied first adjustment.

26. The computer program product of claim 23, wherein the first adjustment comprises a speed of motion of the event marker as the position of the event marker is adjusted.

27. (canceled)

28. (canceled)

29. The computer program product of claim 23, wherein the first adjustment comprises a change in distance of the event marker as the position of the event marker is adjusted.

30. (canceled)

31. (canceled)

32. The computer program product of claim 23, wherein the first adjustment comprises an increase in a number of repetitions of the user interaction with the event marker.

33. The computer program product of claim 23, wherein the first adjustment comprises a decrease in a number of repetitions of the user interaction with the event marker.

34-45. (canceled)

46. A computer program product for clinical evaluation, the computer program product comprising a computer readable storage medium having program instructions embodied therewith, the program instructions executable by a processor to cause the processor to perform a method comprising:
providing a virtual environment to a user via a virtual or augmented reality system;
guiding the user to perform a task involving movement of a body part of the user via the virtual or augmented reality environment, wherein guiding the user to perform the task comprises displaying a visual object to the user;
collecting a first set of data based on the user's performance of the task, the first set of data comprising positional data of the body part;
altering a visual field of the user within the virtual or augmented reality environment;
guiding the user to repeat the task with the altered visual field in the virtual or augmented reality environment;
collecting a second set of data based on the user's performance of the task with the altered visual field, the second set of data comprising positional data of the body part;
providing the first set of data and the second set of data to a remote server via a network;
determining a compliance metric based on the first set of data and the second set of data;
when the compliance metric differs from a predetermined range, applying a first adjustment to the task.

47-53. (canceled)

54. A computer program product for clinical evaluation, the computer program product comprising a computer readable storage medium having program instructions embodied therewith, the program instructions executable by a processor to cause the processor to perform a method comprising:
providing a virtual environment to a user via a virtual or augmented reality system, the virtual environment including an avatar using machine learning or artificial intelligence to communicate with the user;
collecting screening data based on the user's interaction with the avatar in the virtual environment;
determining a customized evaluation, training, or treatment protocol for the user based at least in part on the screening data;
guiding the user to perform a task in the evaluation, training, or treatment protocol via the virtual or augmented reality system;
collecting data from a plurality of sensors relating to the user's performance of the task;
analyzing the data and generating a report based on the performance of the task.
